(19) United States
(12) Patent Application Publication
(10) Pub. No.: US 2019/0041320 A1
(43) Pub. Date: Feb. 7, 2019

(54) Method For Determining In-Situ Suspended Sediment Properties

(71) Applicant: Luxembourg Institute of Science and Technology (LIST), Esch/Alzette (LU)

(72) Inventor: Nuria Martinez-Carreras, Luxembourg (LU)

(21) Appl. No.: 15/760,185

(22) PCT Filed: Sep. 5, 2016
(86) PCT No.: PCT/EP2016/070799

§ 371 Date: Mar. 14, 2018

(30) Foreign Application Priority Data
Sep. 14, 2015 (LU) LU92827

(51) Int. Cl. G01N 21/33; G01N 33/18

(52) U.S. Cl. CPC G01N 21/33; G01N 2201/129; G01N 33/1833

(57) ABSTRACT

A method for determining in situ and in real time at least one suspended sediment property in a medium, the suspended sediment comprising a mineral and an organic fraction. The method comprises the steps of (a) measuring light absorbance with a submersible ultraviolet-visible spectrometer, the ultraviolet-visible spectrometer being configured to analyse to analyse light absorbance at wavelengths which are comprised between 220 nm and 730 nm and of (b) correlating the light absorbance to the properties of the suspended sediment, in various instances by using the Beer-Lambert's law. The method is remarkable in that the step (b) is performed by using one model calibrated for deriving the properties of the suspended sediment from the light absorbance.

### CROSS-REFERENCE TO RELATED APPLICATIONS

The present invention is the US national stage under 35 U.S.C. § 371 of International Application No. PCT/EP2016/070799, which was filed on Sep. 5, 2016, and which claims the priority of application LU 92827 filed on Sep. 14, 2015, the content of which (text, drawings and claims) are incorporated here by reference in its entirety.

### FIELD

The invention is directed to a method to estimate suspended sediment properties, for example the physico-chemical composition of said suspended sediment properties.

### BACKGROUND

Suspended sediments play an important role as a vector for the transfer of nutrients and metals in fluvial systems (Horowitz A. J., Stephens V. C., Sci. Total Environ., 2008, 400, 290-314). For instance, a substantial proportion of the total phosphorus load of many rivers (Walling D., et al. Freshwater contamination, 1997, IAHS Press, Vol. 243, Bowes M. J., Sci. Total Environ., 2003, 313, 199-212) and >90% of the flux of the majority of trace elements (Horowitz A. J., A primer in sediment-trace element chemistry, 1991, 2nd ed., Lewis) are transported in association with fine particles. As a consequence, polluted sediment can lead to undesirable ecosystem conditions and water quality deterioration (Bilotta G. S. and Brazier R. E., Water Res., 2008, 42, 2849-2861). Information on suspended sediment physical and geochemical composition is thus needed in order to implement appropriate sediment control strategies and accomplish legislation requirements.

However, the need to sample suspended sediments, to sieve and to analyse the samples in the laboratory makes the determination of suspended sediment properties very demanding in terms of labour time and cost (Martinez-Carreras N. et al, J. Hydrol, 2010, 382, 49-63; Walling D., J. Soils Sediments, 2013, 13, 1658-1675). With the aim of reducing labour and improving the temporal resolution of suspended sediment properties estimates, spectral reflectance-based methods were developed (Martinez-Carreras N. et al, J. Soil Sediments, 2010, 10, 400-413). These approaches allowed analysing the suspended sediment retained in filter papers after stream water filtration. However, suspended sediment properties can exhibit significant variations within and between storm runoff events, and a robust method for measuring suspended sediment properties in situ, cost efficient, at high-frequency and over large periods of time is still lacking.

Instruments for monitoring suspended sediment properties in situ are limited to sediment physical properties (e.g. particle size characteristics; Walling D. et al, Water Res., 1993, 27, 1413-1421). Contrarily, affordable field-deployable autoanalysers are a reality to measure stream flow chemistry. Some analysers use ion-specific electrodes, while others are based on proxy measurements. One newly available technology based on proxy data are portable ultraviolet-visible (UV-VIS) light spectrometers (220-730 nm). These spectrometers measure the light absorption spectra directly submersed in liquid media. They are often used in wastewater drainage systems for a fast and simple evaluation of wastewater quality (Brito R. S. et al., Urban Water J., 2014, 11, 261-273). Recently, they have been used to estimate turbidity, DOC (dissolved organic carbon) and TOC (total organic carbon) (Avagyan A. et al., J. Hydrol, 2014, 517 435-446), and NO₃—N (Huebsch M. et al., Hydrol. Earth. Syst. Sci., 2015, 19, 1589-1598) in stream at a temporal resolution as high as several minutes.

The hypothesis is that submersible UV-VIS spectrometers can be used to estimate suspended sediment properties in real time. This will move the field forward towards in situ high temporal resolution measurements of suspended sediment properties. To the inventor's knowledge, there is only one study that used a spectrometer to solely estimate suspended sediment properties (i.e. excluding the dissolved fraction) (Bass A. M. et al., Limnol Oceanogr., 2011, 56, 2282-2292). The authors estimated particulate organic matter (POC) at a high temporal resolution and in a catchment. Nonetheless, no calibration of the instrument was performed to predict POC directly. POC was estimated as the difference between TOC and DOC. TOC and DOC were estimated using the internal calibration procedure of the instrument.

### SUMMARY

The invention has for technical problem to provide a method for determining in situ suspended sediment properties which solves all the above explicated problems found in prior art techniques and explicated above. In particularly, traditional ex situ laboratory procedures for measuring suspended sediment physico-chemical properties are generally time-consuming and expensive. For these reasons, the analyses are normally restricted to a relatively low number of samples, which is a major drawback in the precise investigation of the suspended sediment properties.

The invention is directed to a method for determining in situ and in real time at least one suspended sediment property in a medium, the suspended sediment comprising a mineral and an organic fraction. The method comprises the steps of (a) analysing light absorbance with a submersible ultraviolet-visible spectrometer, the ultraviolet-visible spectrometer being configured to analyse light absorbance at wavelengths which are comprised between 220 nm and 730 nm and of (b) correlating the light absorbance to the properties of the suspended sediment, in various instances by using the Beer-Lambert's law. The method is remarkable in that the step (b) is performed by using one model calibrated for deriving the properties of the suspended sediment from the light absorbance.

According to various embodiments, the model has been calibrated from at least one predictor variable obtained through ex situ physico-chemical analysis of the suspended sediment or theoretically, the step of calibration being performed at least once before and/or after the step (a), in various instances before the step (a).

According to various embodiments, the model is a statistical model, in various instances a regression model.

According to various embodiments, the regression model is chosen from ordinary least-square (OLS) regression, robust regression, principal component regression, partial least-squares (PLS) regression, Ridge regression, Lasso regression or non-linear regression.

According to various embodiments, the at least one suspended sediment property comprises the properties of the mineral, in various instances trace elements, and/or of the organic fraction, in various instances polycyclic aromatic hydrocarbons.

According to various embodiments, the at least one suspended sediment property is chosen among total phosphorus concentration, total nitrogen concentration, total carbon concentration, acid-extractable elemental concentrations of Cr, Cu, Ni, Pb and Zn, and/or any other, and/or any combination thereof.

According to various embodiments, the step of analysing light absorbance with a submersible ultraviolet-visible spectrometer is performed, in various instances continuously, with an interval comprised between 1 second and 6 hours between each measurement, in various instances with an interval of 15 minutes between each measurement.

According to various embodiments, the submersible ultraviolet-visible spectrometer is configured to provide one or more of light absorbance measurements, in various instances at 1 nm or 2.5 nm intervals.

According to various embodiments, the submersible ultraviolet-visible spectrometer comprises at least one measuring window and at least one automatic cleaning means configured for automatically cleaning the at least one measuring window.

According to various embodiments, the medium is chosen among a predetermined catchment, a waterway, a river, a stream, a wastewater drainage system, a lake, a water reservoir and/or any other.

According to various embodiments, the step of calibration is repeated at least once, twice, three times, or more, before and/or after step (a), in various instances before step (a).

According to various embodiments, the suspended sediments are extracted from the medium, in various instances by filtration, decantation, freeze-drying and/or wet-drying, before performing the ex situ physico-chemical analysis.

According to various embodiments, the ex situ physico-chemical analysis is performed on the suspended sediments by laboratory analytical methods, in various instances by (a) loss-on-ignition studies, (b) inductively coupled plasma (ICP) spectrometry coupled with an atomic emission spectrometer (ICP-AES) and/or with a mass spectrometer (ICP-MS), and/or atomic absorption spectroscopy (AAS) after applying one or several digestion techniques to the suspended sediment sample, (c) gas chromatograph coupled with mass spectrometer, (d) combustion techniques coupled with titration, gravimetric, manometric, spectrophotometric and/or gas chromatographic techniques, (e) cold vapour atomic absorbance mercury analyser, (f) X-ray diffractometry, (g) differential thermal analysis, and/or (h) any other.

According to various embodiments, the step of calibration further comprises the steps of (a) finding the optimal light absorbance wavelength to use for the model, (b) finding the optimal data transformation; and (c) finding the model coefficients.

According to various embodiments, the at least one predictor variable is chosen among total phosphorus concentration, total nitrogen concentration, total carbon concentration, acid-extractable elemental concentrations of Cr, Cu, Ni, Pb and Zn, and/or any other, and/or any combination thereof.

The invention is particularly interesting in that the described innovative method allows the estimation of suspended sediment physico-chemical properties at high temporal resolution and for long spans of time. Furthermore to be performed directly in the medium, the method is simple, non-destructive and relatively cheap. The method of the present invention could be implemented in different environmental monitoring studies, in hydrological research and/or in water treatment plants.

### DRAWINGS

FIG. 1 illustrates a schematic representation of the method according to various embodiments of the invention.

FIG. 2 illustrates a discharge and rainfall at the Weierbach catchment during the sampling period (December 2013-January 2015) and suspended sediment concentration data (n=47). Precipitation data measured at the Roodt meteorological station, which is located ˜3.5 km south-east from the catchment outlet.

FIG. 3 illustrates a scatter plot and regression line between turbidity and suspended sediment concentration after removal of 5 light absorbance outliers (i.e. spectra with light absorbance values >60% or <2% for all measured wavelengths), 3 turbidity outliers (i.e. turbidity values>60 NTU), and after removal of five other samples that weakened the liner relationship (n=34).

FIG. 4 illustrates a light absorbance spectrums of the calibration set (n=34) and adjusted coefficient of determination (r²) between LOI and absorption at all measured wavelength. Adjusted r² penalizes for higher p.

FIG. 5 illustrates the results obtained with different regression models. m: number of light absorbance wavelengths in the final model; Comp.: optimum number of PLS components that minimises the average standard error of prediction (SEP); r²: coefficient of determination; r²_{adj}: adjusted coefficient of determination.

FIG. 6 illustrates a comparison of measured and predicted LOI values using robust MM-regression. Solid line shows regression between measured and predicted LOI. Dashed line shows 1:1 line.

FIG. 7 illustrates a discharge, rainfall, measured (red diamonds) and predicted LOI from light absorbance data at the Weierbach catchment for the event occurred in December 2014. Predicted LOI estimated using the robust regression calibration method. Erroneous spetroanalyser measurements associated to a dirty measuring window were discarded.

### DESCRIPTION

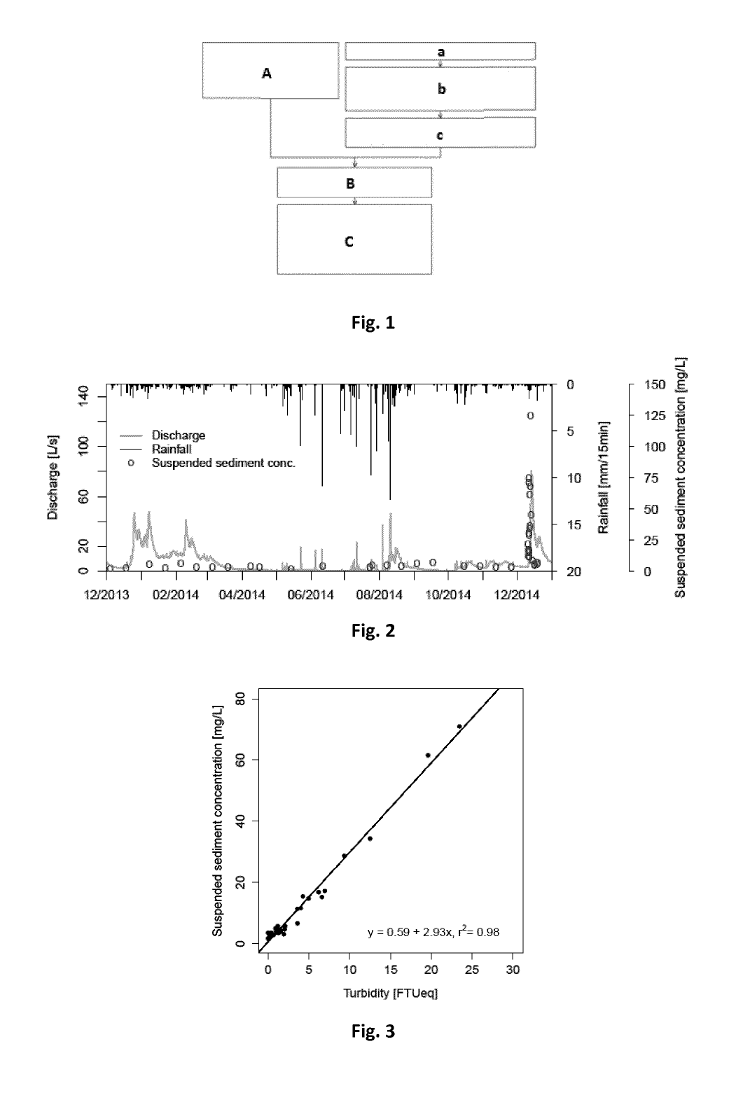

The method of the present invention is described on FIG. 1, in accordance with various embodiments.

On the block A of the schematic representation, in situ water light absorbance measurements are performed at high temporal resolution, for example at 15 minutes intervals. These in situ measurements have an interesting advantages, in time and in cost, compared to the traditional methods which required (a) to limit the collection of medium samples (e.g. stream water) (block a), (b) to separate the suspended sediment from the medium (e.g. stream water) by performing successive steps of filtration, decantation, freeze-drying and/or wet-drying (block b), and (c) to perform traditional laboratory chemical and/or physical analysis of the resulting sediment (block c).

These in situ measurements are achieved by portable submersible ultraviolet-visible light spectrometers (configured to analyse a light with a wavelength which is comprised between 220 nm and 730 nm). They operate in the field directly submerged into the water. They measure the intensity attenuation of a light beam emitted by a lamp after contact with the medium (e.g. stream water) compared to the intensity attenuation when passing through a reference medium (e.g. distilled water). Using the Beer-Lambert's law, it is possible to relate the attenuation of the light to the properties of the medium through which the light is travelling. Specifically, the absorbance of a medium is directly proportional to (a) its thickness (i.e. path length), and (b) to the concentration of the attenuating species in the medium sample.

Chemometric techniques (i.e. the implementation of multivariate data analysis to chemistry-related data) can then be used to determine the concentration of a compound in a complex mixture. Hence, chemometrics aim at developingstatistical models to optimally predict a property y from several variables x₁, x₂, . . . xₙ. y is the property to be estimated and x₁, x₂, . . . xₙ are the light absorbance measurements at different wavelengths.

As suspended sediment comprises a mineral and an organic fraction, the properties of the mineral (e.g. trace elements) and the properties of the organic fraction (e.g. polycyclic aromatic hydrocarbons) might weaken the light beam of the spectrometer in a linear manner. This allows the possibility to calibrate water light absorbance values to predict the physical (e.g. colour) and/or the chemical properties of the suspended sediment.

On the block B of the schematic representation, a calibration step using a statistical model, in particular a regression model, is performed. This kind of step can also be achieved from theoretical data of the suspended sediment which is analysed.

The final part of the method, depicted on the block C of the schematic representation, is the determination of suspended sediment chemical or physical properties at 15 minutes time steps using the statistical model mentioned in the previous paragraph.

The suspended sediment chemical properties that have been studied are mainly total phosphorus concentration, total nitrogen concentration, total carbon concentration, acid-extractable elemental concentrations of Cr, Cu, Ni, Pb and Zn, and/or any combination thereof.

Further details on the method of the present invention are given below, in accordance with various embodiments.

To test the hypothesis, a spectroanalyser (scan, scan Messtechnik GmbH, Vienna) was installed at the outlet of the Weierbach catchment (0.45 km², forested, NE Luxembourg). Stream water light absorbance was measured from December 2013 to January 2015. As a proof-of-concept study, these experimental data were used to calibrate a regression model that predicts suspended sediment loss-on-ignition (LOI) from light absorbance data. LOI was selected because it can be measured in the sediment retained in filter papers after stream water filtration.

Study Area

The Weierbach catchment (0.45 km², 49°49′ N 5°47′ E) is located in the north-western part of Luxembourg. It is mainly covered by mixed Oak-Beech deciduous forest (76% of the land cover; Fagus sylvatica and Quercus petraea) and conifers (24% land cover; Pseudotsuga menziessii and Picea abies). Geology is dominated by Devonian schists, phyllades and quartzite. Soils are shallow (<1 m) and dominated by cambisols, lithosols and colluvisols.

Material and Methods

Spectrometer

The spectroanalyser probe (scan Messtechnik GmbH) was installed at the catchment outlet submersed in the stream. The sensor emits a light beam by a xenon flash lamp. After contact with the stream water, a detector measures the intensity of the beam over the UV-VIS wavelength range.

The sensor was placed at 10 cm above the ground and fixed to a metallic plate to ensure its parallel position to the stream bed during the measurements. This ensures that there is no sedimentation of particles in the measuring section and avoids adhesion of gas bubbles. Three 12 V batteries connected in parallel supplied power. This setup allowed two weeks of continuous measurements at 15 minutes intervals without maintenance.

The scan spectrometer provided measurements of light absorbance at 2.5 nm intervals, as well as turbidity, nitrate concentration, and/or dissolved organic concentration (DOC) using a global calibration developed by the company. However, local calibrations are encouraged by the manufacturer to ensure that parameters are adapted to local concentrations.

The measuring window was automatically cleaned every three hours with compressed air (scan Messtechnik GmbH), and manually cleaned at bi-weekly intervals. Nevertheless, light absorbance spectra showed unrealistic values at some periods of time, most probably due to a dirty measuring window. These unrealistic values (i.e. spectra with light absorbance values >60% or <2% for all measured wavelengths) were discarded. Further, spectra associated to turbidity values >60 NTU were discarded, as considered as unrealistic in the Weierbach catchment.

Stream Water Sampling and Suspended Sediment Concentration Measurements

Stream water samples (1-L) were collected at the outlet of the Weierbach catchment manually at fortnightly intervals from December 2013 to January 2015 (n=24). Additionally, samples were collected during a winter storm runoff event in December 2014 at 1-6 hours intervals (n=23) using automatic water samplers (ISCO 3700 FS and 6712 FS) triggered by water level.

Suspended sediment concentration was determined by filtering a known sub-sample volume (normally between 150 and 250 ml) through 1.2 μm WHATMAN GF/C glass fibre filters by means of a Millipore vacuum pump. The filters were previously dried at 105° C. for more than two hours, cooled in a desiccator, and weighted. After filtration, the filters were dried again at 105° C. and reweighted. The differences between weightings provided the total amount of sediment retained in the filters. The concentration of suspended sediment was calculated by dividing the total amount of sediment retained in the filters by the volume of filtered sample.

Loss-On-Ignition

The LOI method refers to the loss of matter in soils and sediments after an ashing treatment. LOI mainly involves organic matter combustion, decomposition of carbonates and removal of structural water from clay minerals. It informs about the physico-chemical composition of the sediment. LOI was used in the study because it is possible to measure it on the suspended sediment that was retained in the glass fibre filters after filtration. Filters were combusted at 550° C. for 2 h in a muffle furnace. The samples were cooled in a desiccator and weighed again to obtain the weight lost-on-ignition. LOI values are presented as percentage weight loss of dry mass.

Calibration of Light Absorbance Measurements with Percentage Weight Loss-On-Ignition

A regression model was calibrated to predict LOI from light absorbance values by finding (i) the optimal light absorbance wavelength(s) to use for the model, (ii) the optimal data transformation, and (ii) the model coefficients. The light absorbance spectra measured were selected at the same time of or at the time closest to the time manual samples were collected. Assuming a univariate model, according to equation 1.

y=X·b+e (Eq. 1)

In equation 1, y is the variable to be modelled, the matrix X contains the predictor variable, the regression coefficients are collected in the vector b and e is the residual vector. An estimate for the spread of the error distribution is the standard error of prediction (SEP), i.e. the standard deviation of the residuals, according to equation 2.

$$SEP = \sqrt{\frac{1}{z - 1} {\sum }_{i = 1}^{z} {( {e}_{i} - \overline{e} )}^{2}} \qquad ( Eq . 2 )$$

wherein ē is the arithmetic mean of the residuals (or bias). SEP is measured in units of y and can be used to compare the performance of different methods.

With the objective of modelling LOI by one or several light absorbance values, the prediction performance of different linear regression models was estimated with one or several variables (i.e. light absorbance at different wavelengths). Three different regression methods were tested: (1) ordinary least-square (OLS), (2) MM-robust, and (3) partial least-squares (PLS) regressions. In robust statistics, another function than the sum of all squared residuals is minimized, with the objective to diminish the influence of outliers. In PLS regression, instead of minimizing only the explained variance of the predictor variables, PLS components take into account the dependent variables by maximizing, for example, the covariance between the scores of the predictor and modelled variables. This means that PLS components are relevant for the predication of the modelled variable, not for the modelling of the predictors. Several pre-processing techniques were tested but not used as any significant improvement in the predictions was observed (results not shown). Models with multiple variables are fitted to mean-centered data. Moreover, as the LOI data set was rather small, a resampling strategy was used rather than splitting the samples in several sets (i.e. training, validation and test sets) for calibration.

R for statistical analysis (R Core Team, 2013) was used. The R package ‘chemometrics’ implemented by Varmuza and Filzmoser (2009) and its associated vignette (Garcia 200 and Filzmoser, 2015) were used.

Results

Suspended Sediment Concentration and Loss-On-Ignition Measurements

FIG. 2 depicts that the fortnightly sampling covered various flow stages. The maximum sampled suspended sediment concentration during the observation period was 124 mg/L. This coincided with the maximum catchment discharge during the study period (80 L/s). Suspended sediment concentration (SSC) positively linearly correlated to loss-on-ignition (LOI; n=47, r²=0.85, p<0.01). LOI measured values ranged from 1.9 to 9.6%.

Calibration of Light Absorbance Measurements with Percentage Weight Loss-On-Ignition

The performance of a sample set was studied to calibrate the relationship between LOI and light absorbance. This sample set resulted from the exploration of the relationship between suspended sediment concentration and turbidity (FIG. 3). A scatter plot and a regression line between turbidity and suspended sediment concentration, after removal of 5 light absorbance outliers (i.e. spectra with light absorbance values >60% or <2% for all measured wavelengths), 3 turbidity outliers (i.e. turbidity values >60 NTU), and after removal of five other samples that weakened the liner relationship (n=34) are shown.

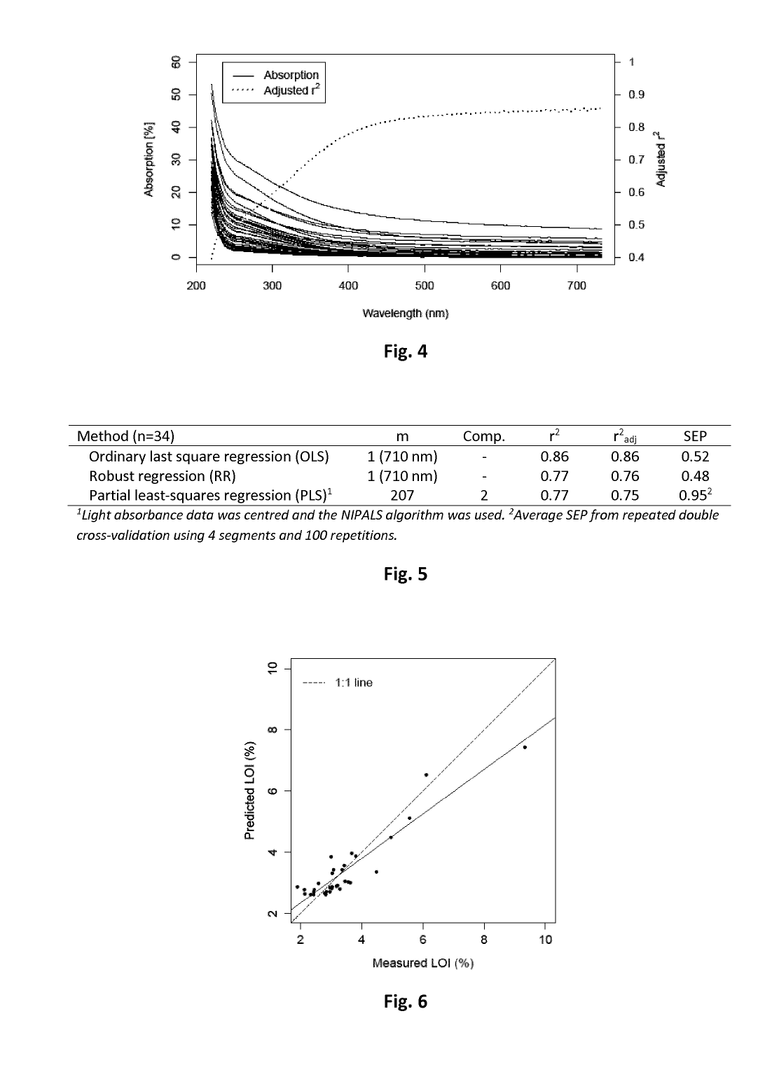

Ordinary least-square regression between LOI and light absorbance was performed at all measured wavelengths (220-735 nm). Measured absorption spectrum for the calibration set and adjusted r² results are shown in FIG. 4. Results show that wavelengths in the visible part of the spectra (i.e. 400-735 nm) have a higher correlation to LOI (up to 0.86 adjusted r² for the calibration set, at 710 nm), compared to the ultraviolet range (i.e. 100-400 nm). The standard error of prediction (SEP) for calibration set was 0.52% (see FIG. 5).

The resulting coefficients of determinations for the MM-robust regression between LOI and light absorbance at all measured wavelengths (220-735 nm) were lower for calibration set to those obtained using OLS regression (maximum of 0.77 at 710 nm, see FIG. 5). However, the SEP was lower (i.e. 0.48% at 237 nm). FIG. 6 shows the scatterplot between measured and predicted LOI values (from light absorbance at 710 nm) using MM-robust regression for the calibration set.

The partial least-squares regression method with repeated double cross-validations (100 predictions for each measured LOI) resulted in higher average SEP value (0.95% for the calibration set) and lower coefficients of determination (0.77 for calibration set) than with the other two methods (see FIG. 5). Moreover, the 100 predictions of LOI for each measured value presented relatively high dispersions (data not shown), resulting in relatively high uncertainty ranges.

Prediction of Percentage Weight Loss-On-Ignition from Light Absorbance Measurements

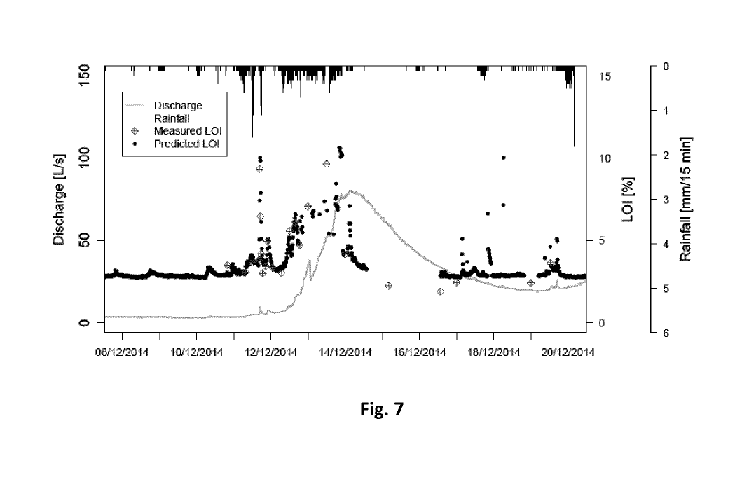

MM-regression was used to predict LOI from light absorbance at 710 nm as the predicted error of prediction was lower than with the other methods (see FIG. 5). FIG. 7 shows measured and predicted LOI from light absorbance data collected at 15-min intervals at the Weierbach catchment for the storm runoff event occurred in December 2014.

Stream flow response during the event was double peaked. Specifically, several small stream flow peaks occurred and their timing was coincident with the rainfall inputs. Later on, a larger and delayed peak occurred. Higher suspended sediment LOI percentages were measured during the relatively low discharge peak occurred the 11 December and during the rising limb of the hydrograph (i.e., 12 and 13 December). During the long falling limb (i.e. from 14 to 18 December) LOI percentages decreased to pre-event values.

Discussion on Predicting LOI from Light Absorbance Data

Several calibration methods were tested to predict LOI from light absorbance (i.e. obtained in-situ) and relatively good correlation were found between predicted values (i.e. obtained ex-situ) and measured values (i.e. obtained in-situ) (see FIG. 5).

When using MM-regression for calibration, a good agreement between predicted and measured LOI was observed (FIG. 7). This could be expected as measured samples were included in the calibration set due to the low number of available measurement. The predictive model did not predict LOI values lower than 2.7% (FIG. 6). This might be due to the low concentration of sediment in stream water when such a low LOI values occurred. Consequently, this sediment might have a very low impact on the light absorbance measurements.

During the studied storm runoff event (FIG. 7), the increase in LOI values during the rising limb of the hydrograph might be associated to a remobilization of suspended sediments enriched in organic matter from the stream and/or near stream areas.

1-15. (canceled)

16. A method for determining in situ and in real time at least one suspended sediment property in a medium, the suspended sediment comprising a mineral and an organic fraction, said method comprising the following steps:
a) analyzing light absorbance with a submersible ultraviolet-visible spectrometer, the ultraviolet-visible spectrometer being configured to analyze light absorbance at wavelengths that are between 220 nm and 730 nm; and
b) correlating the light absorbance to the properties of the suspended sediment, wherein,
the step (b) is performed by using one model calibrated for deriving the properties of the suspended sediment from the light absorbance.

17. The method according to claim 16, wherein the step (b) of correlating the light absorbance to the properties of the suspended sediment is performed by using the Beer-Lambert's law.

18. The method according to claim 16, wherein the model has been calibrated from at least one predictor variable obtained through ex situ physico-chemical analysis of the suspended sediment or theoretically, the step of calibration being performed at least one of at least once before the step (a) and at least once after the step (a).

19. The method according to claim 16, wherein the model is a statistical model.

20. The method according to claim 16, wherein the model is a regression model.

21. The method according to claim 20, wherein the regression model is chosen from one of ordinary least-square (OLS) regression, robust regression, principal component regression, partial least-squares (PLS) regression, Ridge regression, Lasso regression or non-linear regression.

22. The method according to claim 16, wherein the at least one suspended sediment property comprises the properties of at least one of the mineral and of the organic fraction.

23. The method according to claim 22, wherein the at least one suspended sediment property comprises trace elements.

24. The method according to claim 22, wherein the at least one suspended sediment property comprises polycyclic aromatic hydrocarbons.

25. The method according to claim 16, wherein the at least one suspended sediment property is chosen among at least one of total phosphorus concentration, total nitrogen concentration, total carbon concentration, acid-extractable elemental concentrations of at least one of Cr, Cu, Ni, Pb and Zn, and any other, and any combination thereof.

26. The method according to claim 16, wherein the step of analyzing light absorbance with a submersible ultraviolet-visible spectrometer is performed with an interval between 1 second and 6 hours between each measurement.

27. The method according to claim 16, wherein the submersible ultraviolet-visible spectrometer is configured to provide one or more of light absorbance measurements.

28. The method according to claim 16, wherein the submersible ultraviolet-visible spectrometer comprises at least one measuring window and at least one automatic cleaning means configured for automatically cleaning the at least one measuring window.

29. The method according to claim 16, wherein the medium is chosen among at least one of a predetermined catchment, a waterway, a river, a stream, a wastewater drainage system, a lake, a water reservoir and any other.

30. The method according to claim 16, wherein the step of calibration is repeated at least once, twice, three times, or more, at least one of before and after step (a).

31. The method according to claim 18, wherein the suspended sediments are extracted from the medium before performing the ex situ physico-chemical analysis.

32. The method according to claim 18, wherein the ex situ physico-chemical analysis is performed on the suspended sediments by laboratory analytical methods.

33. The method according to claim 32, wherein the laboratory analytical methods are at least one of:
a) loss-on-ignition studies;
b) inductively coupled plasma, ICP, spectrometry coupled with an atomic emission spectrometer, ICP-AES, and/or with a mass spectrometer, ICP-MS, and/or atomic absorption spectroscopy, AAS, after applying one or several digestion techniques to the suspended sediment sample;
c) gas chromatograph coupled with mass spectrometer;
d) combustion techniques coupled with titration, gravimetric, manometric, spectrophotometric and/or gas chromatographic techniques;
e) cold vapour atomic absorbance mercury analyser;
f) X-ray diffractometry;
g) differential thermal analysis, and
h) any other.

34. The method according to claim 18, wherein the step of calibration further comprises the steps of:
a) finding the optimal light absorbance wavelength to use for the model;
b) finding the optimal data transformation; and
c) finding the model coefficients.

35. The method according to claim 18, wherein the at least one predictor variable is chosen among at least one of total phosphorus concentration, total nitrogen concentration, total carbon concentration, acid-extractable elemental concentrations of at least one of Cr, Cu, Ni, Pb and Zn, and any other, and any combination thereof.
